(19) United States
(12) Patent Application Publication
(10) Pub. No.: US 2016/0324570 A1
(43) Pub. Date: Nov. 10, 2016

(54) Injection Coagulation Electrosurgical Device

(71) Applicant: Hamid Ehsani-Nia, Northridge, CA (US)

(72) Inventor: Hamid Ehsani-Nia, Northridge, CA (US)

(21) Appl. No.: 15/211,216

(22) Filed: Jul. 15, 2016

(51) Int. Cl. A61B 18/14; A61B 17/00

(52) U.S. Cl. CPC A61B 18/1477; A61B 17/00491; A61B 2017/005; A61B 2018/00589

(57) ABSTRACT

A electrosurgical and injection device is combination disclosed herein allows for one to inject fluid, such as a protein or synthetic solution, and coagulate and/or solidify this solution as well as nearby tissue. At an extreme end of a generally elongated device is a tip or business end with a portal leading into a conduit where the fluid passes through before exiting out of the tip. Around this portal, on the tip are one (monopolar) or two (bipolar) electrodes used for coagulation.

### FIELD OF THE DISCLOSED TECHNOLOGY

The disclosed technology relates generally to devices for electrosurgery, and more specifically to a device which injects fluid into a cavity or anatomical lesion and is able to cut, coagulate, and desiccate tissue, while also coagulating the injected substance from a fluid into a clotted solidified substance.

### BACKGROUND OF THE DISCLOSED TECHNOLOGY

Electrosurgery is carried out on the prior art by passing alternating electric current at desired high frequencies through an electrode or combination thereof that pass the electrical current through the tissues of the body and thus through the cells. The oscillation of this alternating current causes the ions within the cells to physically oscillate rapidly, causing the cells to heat. The modulation of voltage, amperage, and frequency by the operator can thereby cause the desired effects of dessicating or vaporizing cells, and can thus allow the operator to cut tissue or to coagulate the microcellular structures or to induce coagulation, thereby stopping bleeding. This can be done in two ways: monopolar or bipolar. In monopolar a single electrode (for purposes of this disclosure, defined as having a width less than 3 cm and at which electric current jumps to the body) with current is in contact with the body and a much larger second electrode (defined as at least 300% greater in surface area) is used to receive the current. Such a “much larger area” of the second electrode is herein referred to as a “current receiving plate” or “dispersive electrode”. For example, on the thigh a large patch/current receiving plate can be placed over which the electric current is far more diffuse when exiting the body than the small point of an electrode in contact or extremely close thereto a portion of the body being cut or coagulated with electrosurgery. Depending on frequency, body tissue is cut or coagulated. In bipolar electrosurgery the current is passed between two closely placed electrodes having similar size endings.

### SUMMARY OF THE DISCLOSED TECHNOLOGY

An electrosurgical device of embodiments of the disclosed technology includes a fluid reservoir fluidly connected to a hollow conduit and tip with portal. It further has at least one electrode on the tip, and in some embodiments, two electrodes (positive and negative polarity). A pump causes fluid in the reservoir to exit through the portal of the tip. The electrode can be circular in shape, or with two electrodes, they can each form part of the circle but be spaced apart from one another. The fluid reservoir holds a protein or synthetic solution which solidifies or coagulates upon contact with electricity (above a certain threshold of strength and/or of a certain frequency) in embodiments of the disclosed technology.

One can use this device by inserting a part or all of the tip, including the portal located at the tip, into a place (such as lumen of an individual after surgery, or into a bleeding lesion from a traumatic injury) to be coagulated. The pump is then activated to cause fluid in the reservoir to pass through the hollow conduit and out of the portal into the place to be coagulated. While holding the tip substantially at this place to be coagulated (defined as a place close enough to cause coagulation/solidification at the actual place desired for coagulation), and with said at least one electrode in contact with the injected solution (defined as close enough to send a majority of the voltage at the tip itself through the injected solution), an electric current is engaged until at least some of the injected solution solidifies.

The electrosurgical device can further have a handle region situated between the fluid reservoir and the tip. The hollow conduit, in embodiments of the disclosed technology, passes through the handle region. A button operable to activate the pump and a button operable to activate the electric current to the electrode or electrodes can be located on this handle.

The tip can be at a right or acute angle to the elongated body and/or conduit. In some embodiments, at least one electrode surrounds the majority of the hollow conduit. So too, when there are two electrodes, in such an embodiment the two electrodes surround a majority of the conduit with two spaces between the arc-shaped electrodes, in total, being less than a majority thereof in an arc.

Another way of describing electrosurgical devices of embodiments of the disclosed technology is as a housing with fluid conduit situated between a business end and fluid reservoir. An electrode at an extreme tip of the business end (a narrow end, compared the relatively long elongated housing and conduit). A portal enters into the fluid conduit also at the extreme tip of the business end. At least one button on the housing, between the business end and fluid reservoir engages an electrical circuit passing through the housing. Engagement of the electrical circuit causes a pump to push fluid from the reservoir out of the portal or solidification of the fluid which has been pushed out of the portal. These can be two electrical circuits, each doing one of the aforementioned tasks and a button to engage each circuit. The circuits can be engaged one after the other such that, for example, some fluid is released, then solidified, and more fluid is released and solidified by a continuing current applied at the electrode or electrodes. The circuits can be engaged simultaneously so that the fluid is constantly being coagulated as soon as it exits the lumen.

Any device or step to a method described in this disclosure can comprise, or consist of, that of which it is a part, or the parts which make up the device or step. The term “and/or” is inclusive of the items which it joins linguistically and each item by itself.

### BRIEF DESCRIPTION OF THE DRAWINGS

FIG. 1 shows a front and side view of a monopolar electrosurgery device of embodiments of the disclosed technology.

FIG. 2 shows a front and side view of a bipolar electrosurgery device of embodiments of the disclosed technology.

FIG. 3 shows a side elevation view of a monopolar electrosurgery device of embodiments of the disclosed technology.

FIG. 4 shows a side elevation view of a bipolar electrosurgery device of embodiments of the disclosed technology.

FIG. 5 shows a detail of the tip of the monopolar electrosurgery device of FIG. 1.

FIG. 6 shows a detail of the rear end of the monopolar electrosurgery device of FIG. 1.

FIG. 7 shows a detail of the tip of the bipolar electrosurgery device of FIG. 2.

FIG. 8 shows a detail of the rear end of the bipolar electrosurgery device of FIG. 2.

FIG. 9 shows a path for injection of solution using an electrosurgery device of embodiments of the disclosed technology.

FIG. 10 shows a path for electrical current within an electrosurgery device of embodiments of the disclosed technology.

FIG. 11 shows a path for electrical current in a monopolar electrosurgery device of embodiments of the disclosed technology.

FIG. 12 shows a path for electrical current in a bipolar electrosurgery device of embodiments of the disclosed technology.

FIG. 13 shows cross sections of the monopolar electrosurgery device shown in FIG. 1.

FIG. 14 shows cross sections of the bipolar electrosurgery device shown in FIG. 2.

### DETAILED DESCRIPTION OF EMBODIMENTS OF THE DISCLOSED TECHNOLOGY

An electrosurgery and injection device is combination disclosed herein allows for one to inject fluid, such as a protein solution or synthetic solution, and coagulate and/or solidify this solution as well as nearby tissue. At an extreme end of a generally elongated device is a tip or business end with a portal leading into a conduit where the fluid passes through before exiting out of the tip. Around this portal, on the tip are one (monopolar) or two (bipolar) electrodes used for electrosurgery.

Embodiments of the disclosed technology are described below, with reference to the figures provided.

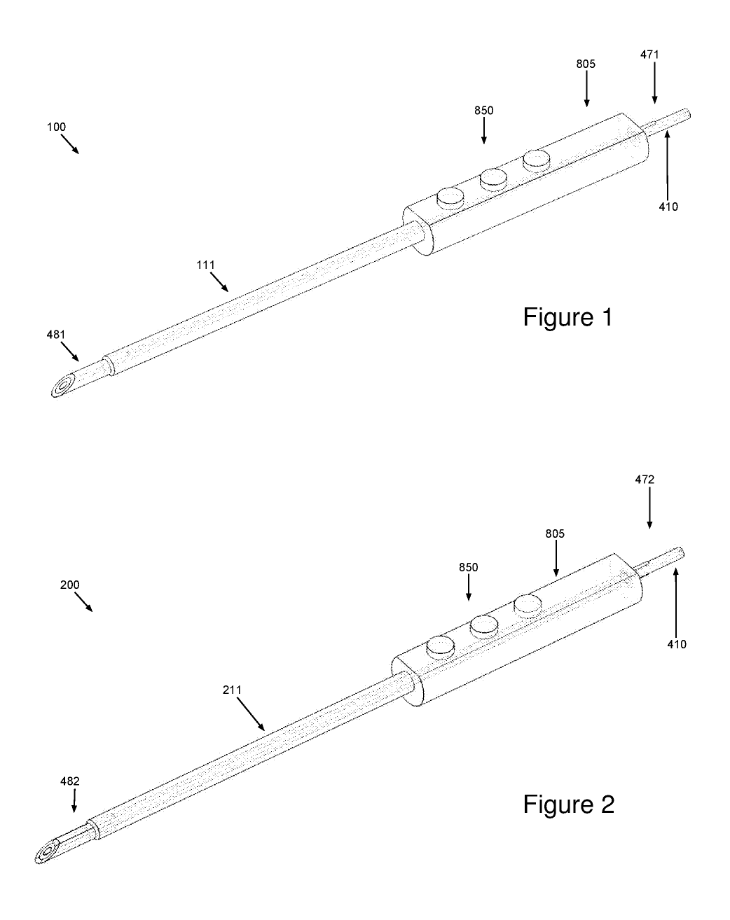

FIG. 1 shows a front and side view of a monopolar electrosurgery device of embodiments of the disclosed technology. A monopolar electrosurgery device 100 is shown. Monopolar, for purposes of this disclosure, is defined as a device with a single electrode at a business end of the electrosurgery device which comes in contact with tissue and injected fluid and/or by which electrical current exits the device and coagulates the tissue. A second much larger area of conductive material is in contact with another part of the body and is used for the exit of electrical current as the second electrode of the alternating current circuit. Tissue refers to living or a formerly living collection of cells in a human or animal body.

FIG. 2 shows a front and side view of a bipolar electrosurgery device of embodiments of the disclosed technology. A bipolar electrosurgery device 200 is shown. Bipolar, for purposes of this disclosure is defined as having two electrodes at a business end of the electrosurgery device which each either come in contact with tissue or injected fluid and/or send or receive electrical current that will pass through or has passed through tissue or injected fluid to be coagulated.

Elements of the bipolar device is incremented by 100 compared to elements of the monopolar device. Discussing both FIGS. 1 and 2 simultaneously, each comprises an injection fluid reservoir apparatus 401 which can have a pump 870. This reservoir comprises fluid which will be injected into a surgical cavity within a body or other area to be coagulated. This fluid can comprise a protein rich mixture or a synthetic mixture. Upon subjugation of this fluid to electrical current, the protein or synthetic materials coagulate. This coagulated substance can have a specific half life depending on the procedure. The fluid solution can also be hyper/hypotonic depending on the judgment of the operator. A hypertonic solution aids in the conduction of current through the fluid and thus aids in the coagulation. The fluid can also include small amounts of microscopic metal particles to aid in conduction for the same reason. In any case, the injected fluid is made of substances that will not cause adverse immune reaction by the patient. Additionally, the solution can include antibiotics to be use therapeutically, empirically, or prophylactically. The solution can also include other pharmacological modalities to treat specific conditions the patient is subjected to. Additionally, the solution can include a dye that will make locating it visually apparent in subsequent procedures. A hollow conduit 410 fluidly connects to the fluid reservoir 401 allowing for passage of the injection fluid through the body 805 of the device 100 or 200 as well as through a hollow tip 481 or 482. As such, the direction of injection fluid flow is from the reservoir 401 through a hollow conduit 410 which has a continuous fluid connection all the way to a respective tip 481 or 482. Device control elements 850, such as electrical or mechanical buttons, operate a pump, siphon, or opening to an aperture allowing or causing the injection fluid to flow from the reservoir 401, or other place where fluid is being held, out through the top 481 or 482. These device control elements 850 are also used to open/close as well as modulate the radio frequency alternating electrical current.

Referring still to FIGS. 1 and 2, other elements shown include insulating material 111/112 and 211/212 which cause the hollow conduit 410 to have a thicker outer circular cross-section area than a tip area 481/482 thereof. Likewise, the body of the device can have a thicker housing 805 for easier gripping and control of the device as well as housing mechanical or electrical components of the device including a pump, the reservoir 401, a battery, electrical controls, and so forth. A coupling 471 or 472 can be used to attach the insulating material 111 or 211 to the hollow conduit 410. The lumen of 410 is labeled as 405, and it is a continuation of the same lumen of 210/110.

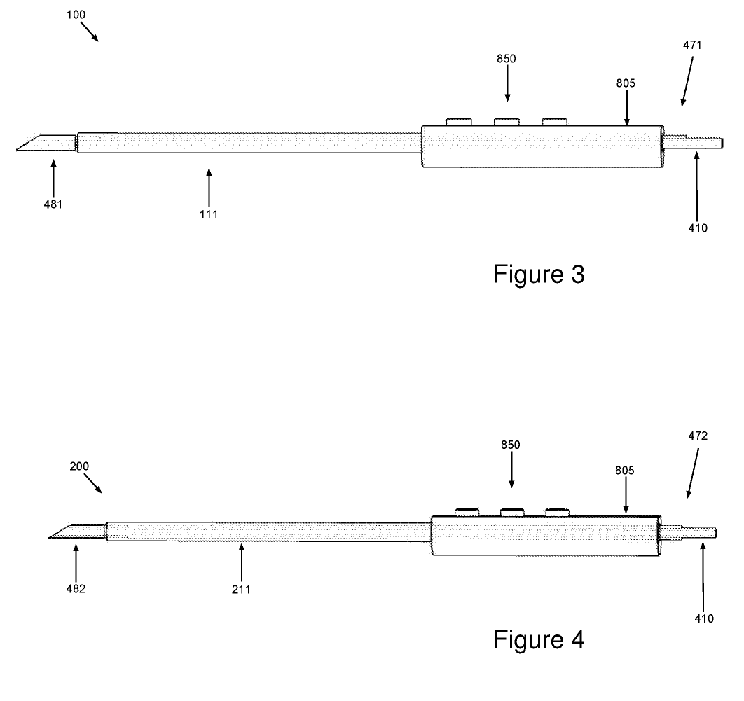

FIG. 3 shows a side elevation view of a monopolar electrosurgical device of embodiments of the disclosed technology. FIG. 4 shows a side elevation view of a bipolar electrosurgical device of embodiments of the disclosed technology. In these side views, the conduit 410 extending from the reservoir 401 is shown extending through, in order, the coupling 471/472, the external insulation 111/211, the housing 805, the front external insulation 111/211, and becoming or ending at a tip 481/482.

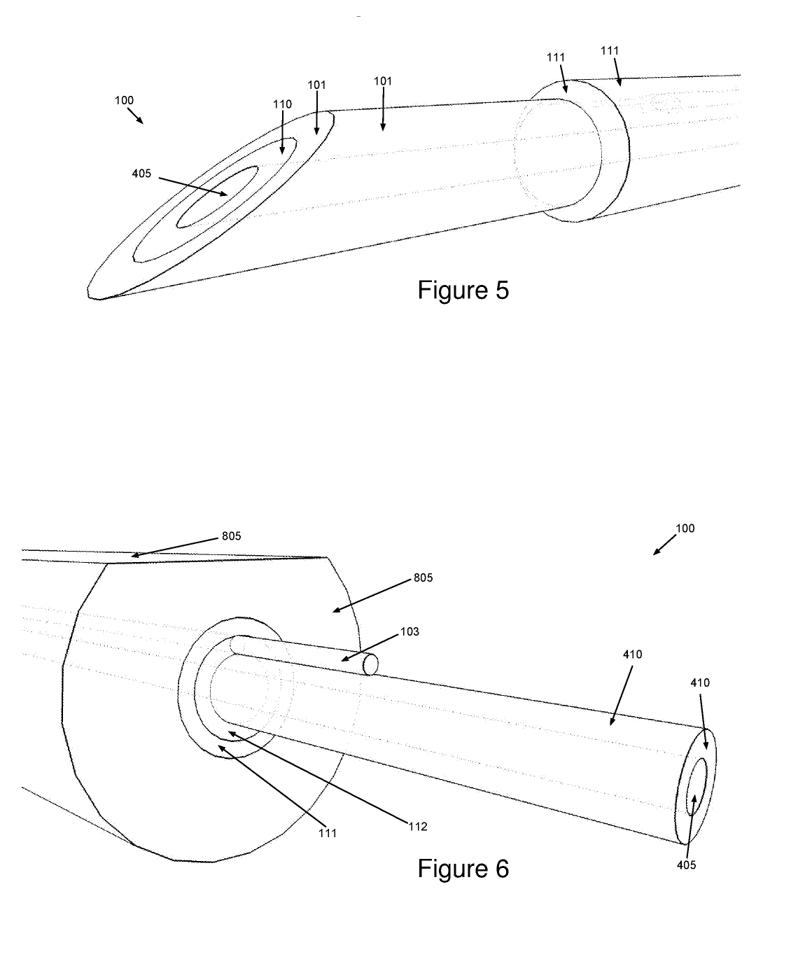

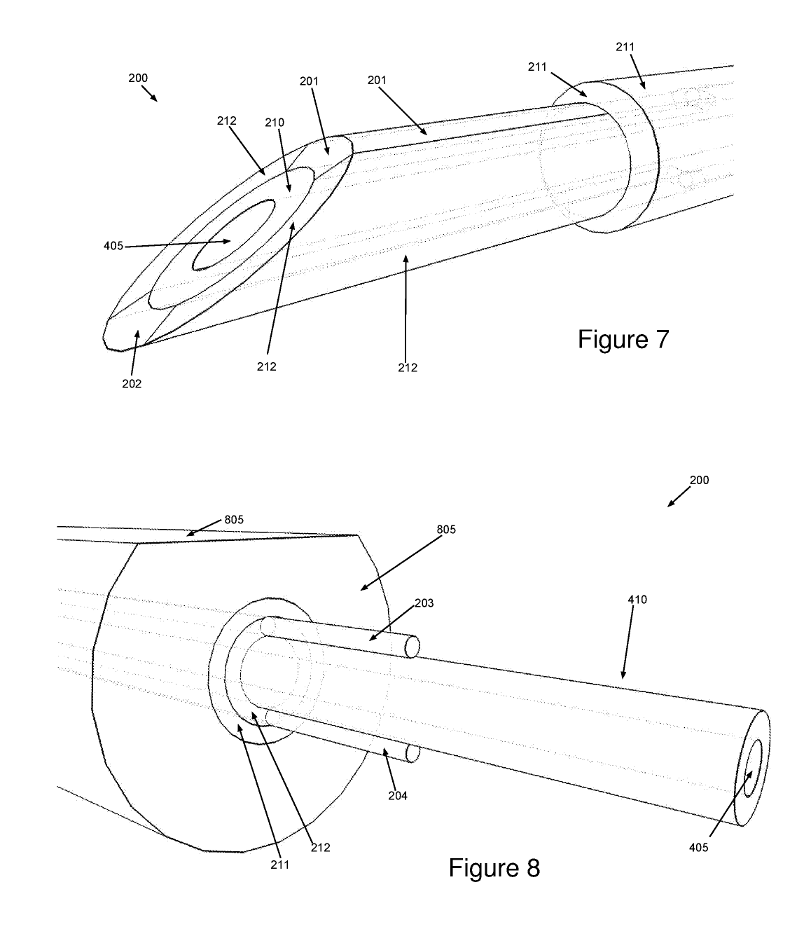

Closeup versions of the tips and rear portions of the electrosurgical devices follow. FIG. 5 shows a detail of the tip of the monopolar electrosurgical device of FIG. 1. FIG. 7 shows a detail of the tip of the bipolar electrosurgical device of FIG. 2. The external insulation 111 or 211 ends and an exposed portion 101 or 201 is hollow and fluidly connected to the reservoir 401 or any other reservoir having some fluid to be injected. The tip 481/482 can have an end which forms a right angle or acute angle to the elongated length of the conduit 410. A business end or front portal 405 into the interior of the hollow conduit 410 allow injection fluid to exit from the device 100/200. The business end or tip further has an extreme end with internal insulating material 110/210. In embodiments of the disclosed technology, a single electrode (monopolar) or two separate electrodes (bipolar). The electrode or electrodes, in embodiments, can be anywhere along the tip which, for purposes of this disclosure, is defined within 10 cm of an extreme end which is a business end of the device 100/200. The portal 405, in alternative embodiments, is on a side of the tip as opposed to at the extreme end, as shown in FIGS. 5 and 7.

FIG. 6 shows a detail of the rear end of the monopolar electrosurgical device of FIG. 1. FIG. 8 shows a detail of the rear end of the bipolar electrosurgical device of FIG. 2. The hollow conduit 410 carries the injection liquid. The conduit 110/210 and 410, though shown in different circumferences can be continuous and of the same circumference in other embodiments. The extreme back end of the insulation 110/210 or coupling is within the insulation 111/211.

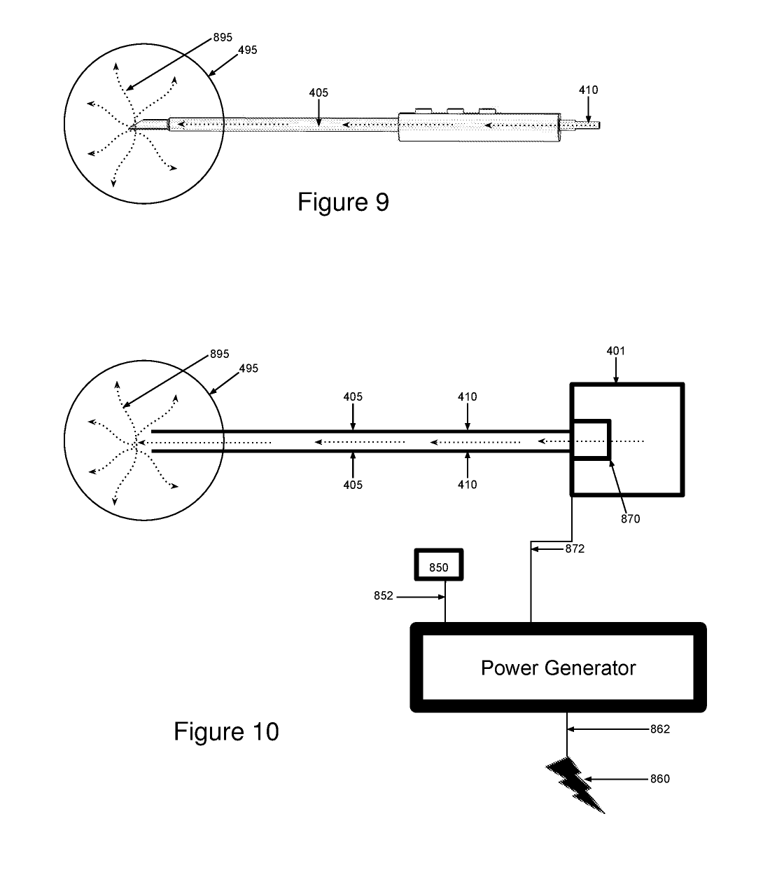

FIG. 9 shows a path for injection of protein using a electrosurgical device of embodiments of the disclosed technology.

FIG. 10 shows an electrical schematic diagram for electrical current within a electrosurgical device of embodiments of the disclosed technology. Current extends from a current source 860 (though the current source can be in the housing 805 or exterior to the device 100/200 entirely) to the pump 870. Before applying current through the electrodes, injection fluid 495 is placed onto tissue or within a cavity of a person such as during a surgical operation. The fluid is expelled out of the hollow tube 405 and the front tip of the electrosurgical device. Then, at the same front tip current is applied. In this manner, one can insert the front tip of the electrosurgical device into the lumen of a person or into a wound or lesion, expel fluid through the front tip, and then without moving the front tip or substantially moving (wherein “substantially” for purposes of this disclosure is defined as “no more than 10 cm”) the front tip, can coagulate the tissue and expelled solution injection fluid 495. The electrical current passes from electrodes of the electrosurgical device through the fluid as well as tissues of the person. The direction of the flow of the fluid after being injected is shown by 895. The subsequent injection bubble formed within the body cavity by the injection of fluid is shown by 495.

In more detail, still referring to FIG. 10, a power source 860 is connected to a power generator by a wire 862. The power generator can be used to modify the frequency or amplitude of the electrical current sent to the electrodes. This can be done by using a switch 850 which can also control when power is sent via the wire 872 to a reservoir for fluid 401 where it is pumped by a pump 870 towards the tip of the electrosurgical device via conduit 405/410. Solution 495 is injected into the lumen of a person which extends outwards from the point of injection in the directions 895.

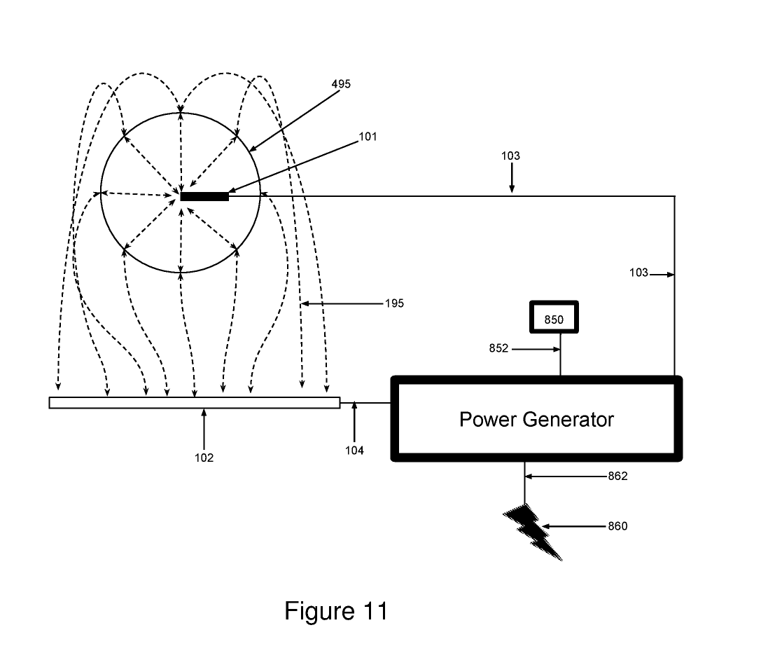

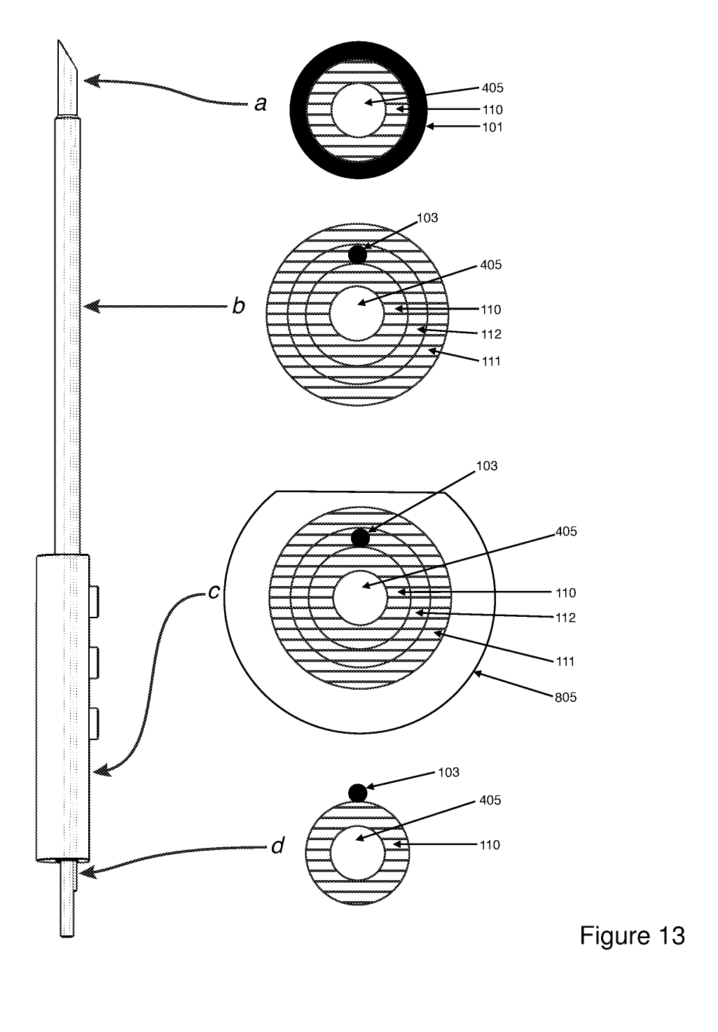

FIG. 11 shows a path for electrical current in a monopolar electrosurgical device of embodiments of the disclosed technology. Electricity is generated and received from an external power source, internal power source, or battery 860 via a wire 862. Controls 850, such as a mechanical or electrical buttons as shown in FIGS. 1 and 2, for example, are electrically or mechanically coupled via a wire or mechanical connection mechanism 852. Electricity is then sent via wire 103 through the body of the electrosurgical device around the hollow area thereof (represented by the exposed portion 101 which contains there-within a hollow interior region for passage of fluid). Via wire 103 situated between the hollow interior space and the insulation (see FIG. 1 or 13), electric current is sent out a node or electrode at the tip of the electrosurgical device 100. Fluid 495 which has already been expelled out of this tip receives the electric current which then solidifies the proteins/synthetic materials in the fluid causing coagulation in the lumen or lesion of a person. The current is dissipated as it continues to flow through the fluid 495 and the body of a person via the path lines 195 shown to demonstrate same, until reaching the current receiving plate 102. This current receiving plate has a conductive surface or a plurality of conductor electrodes (designed for low impedance and easy flow of electric current) which then receives the current back through a wire 104 to the power generator, completing the circuit.

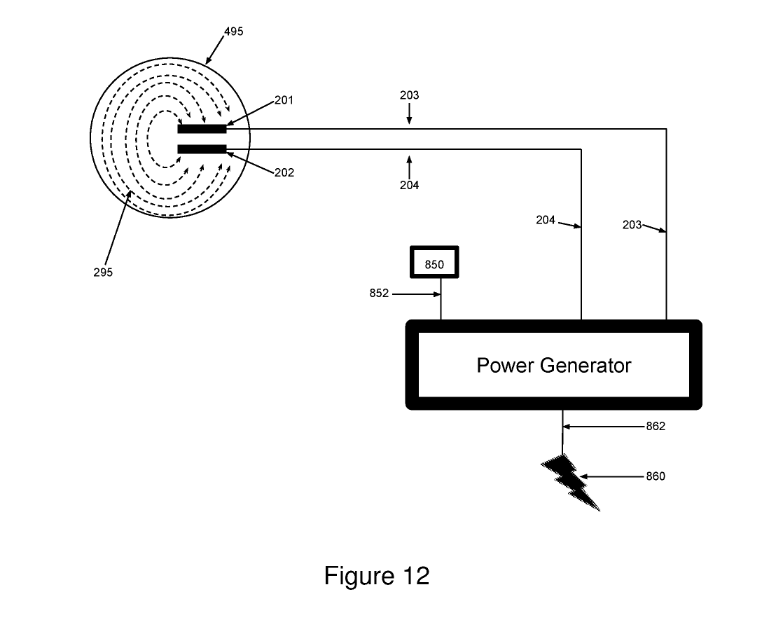

FIG. 12 shows a path for electrical current in a bipolar electrosurgical device of embodiments of the disclosed technology. In this embodiment, the items which are numbered the same as with regards to FIG. 11 should be treated accordingly. Here, there are two electrodes/paths for the current out of the electrosurgical device, namely electrodes 201 and 202 as opposed to the single electrode/path for the current 103 through the monopolar device 100. The fluid 495 is ejected from the device 200 and the current flows from the power source 860 out through wire 203, through the device 200 (such as along inner region 201 of the device) and into the fluid 495 illustrated by lines 295. The current then flows back through the device via a second electrode 202 and a pathway along a different portion of the inner region 202 and wire 204. In this manner, the electric current enters and returns from the fluid 495 and lumen/lesion of a person in close proximity (defined as within 10 cm).

FIG. 13 shows cross sections of the monopolar electrosurgical device shown in FIG. 1. Cross section a is a cross section of the tip, the narrowest region of the device at the business end thereof which is inserted into the patient. The tip region has a maximum length of 1, 5, 10, or 20 cm in different embodiments. In other embodiments, it extends from an extreme tip as far as the circular circumferential width remains constant. At an outermost side of the tip, at the extreme end or a position along the outside of the tip region, is a single electrode 101 supported by the conduit 110 which encloses a portal 405 for passageway of liquid. Conduit 110 is made of insulating material, which prevents the electrical current exiting electrode 101 to enter the lumen 405, and thus prevents the liquid inside 110 from coagulating until it has exited the tip of the device. The next region, shown in cross section b adds an additional outer layer of insulating material 111 such that the part of the conductive metal 101 and electrical connection to wire 103 are unexposed to the outside. Insulating layer 112 serves to insulate as well as physically support wire 103. At cross section c there is a handle with a further layer 805. At cross section d the electrical wire 103 extends from the device and can be connected to the other components illustrated in FIG. 11. In embodiments, the power source is connected here and covered by the an electrical connector. Any method of electrifying the electrode 101 at the business end can be used in addition to the particular arrangement shown in this figure. Also seel is the continuation of hollow inner insulating tube 110, which can subsequently be connected to fluid conduit 410, and that can subsequently be connected to the fluid reservoir apparatus 401 (which is also illustrated in FIG. 10).

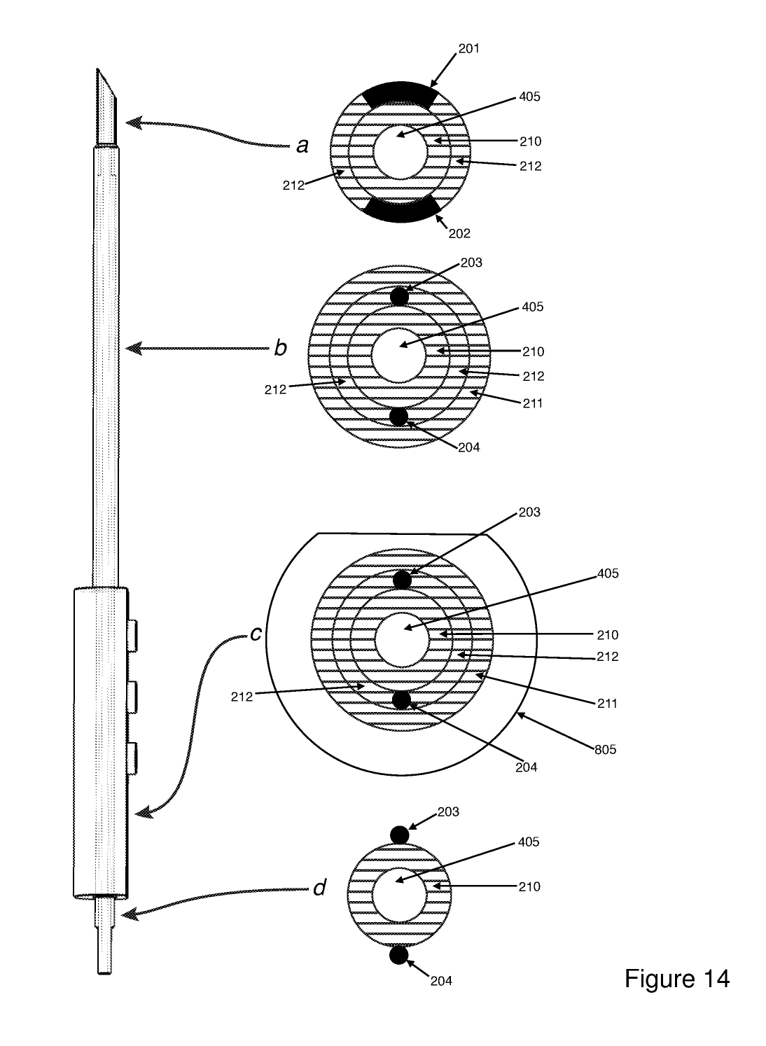

FIG. 14 shows cross sections of the bipolar electrosurgical device shown in FIG. 2. Elements of FIG. 13 has been incremented by 100 and remain as described with reference to FIG. 13. The exception is that the bipolar embodiment has two electrodes 201 and 202 which are spaced apart from each other, such as, as semi-circular ended electrodes. These two electrodes are connected to two wires 203 and 204 (respectively) which can extend as a metal wire in two parts through cross sections a, b, c, and d. Additionally, there is a middle layer of insulation 212 to physically insulate and separate electrodes 201 and 202, as well as their respective wires 203 and 204.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods, systems, and devices described hereinabove are also contemplated and within the scope of the disclosed technology.

I claim:

1. An electrosurgical device comprising:
a fluid reservoir fluidly connected to a hollow conduit and tip with portal;
at least one electrode on said tip;
a pump adapted to cause fluid in said reservoir to exit through said portal of said tip.

2. The electrosurgical device of claim 1, wherein said at least one electrode is two electrodes on said tip, each separate from one other.

3. The electrosurgical device of claim 2, wherein of said two electrodes, one is a positive terminal and the other is a negative terminal.

4. The electrosurgical device of claim 1, wherein said fluid reservoir holds a liquid solution which solidifies upon contact with electricity.

5. A method of using said electrosurgical device of claim 4, comprising the steps of:
inserting said tip with said portal into a place to be coagulated;
activating said pump causing fluid in said reservoir to pass through said hollow conduit out said portal;
while holding said tip substantially at said place to be electrosurgical and with said at least one electrode in contact with said protein solution, engaging an electric current until at least some of said solution solidifies.

6. The device of claim 1, wherein:
a handle region is situated between said fluid reservoir and said tip;
said hollow conduit passes through said handle region; and
a button operable to activate said pump and a button operable to activate electric current to said at least one electrode is located at said handle.

7. The device of claim 6, wherein said tip is at an acute angle to said conduit.

8. The device of claim 1, wherein said at least one electrode surrounds at majority of said hollow conduit.

9. The device of claim 2, wherein said two electrodes, in combination, surround a majority of said hollow conduit.

10. The device of claim 8, wherein said electrode is situated between and in physical contact with two layers of insulation, a first layer of said two layers forming an exterior of said hollow conduit.

11. An electrosurgical device, comprising:
a housing with fluid conduit situated between a business end and fluid reservoir;
an electrode at an extreme tip of said business end;
a portal entering into said fluid conduit also at said extreme tip of said business end;
at least one button on said housing, between said business end and said fluid reservoir engages an electrical circuit passing through said housing.

12. The electrosurgical device of claim 11, wherein said engagement of said electrical circuit causes a pump to push fluid from said reservoir out said portal.

13. The electrosurgical device of claim 12, wherein engagement of a second electrical circuit using a second button on said housing causing solidification of said fluid which has been pushed out of said portal.

14. The electrosurgical device of claim 13, wherein some of said fluid is pushed out of said portal before said second button is engaged and more said fluid is pushed out of said portal while said second button is engaged.

15. The electrosurgical device of claim 14, wherein said electrode is two separate electrodes at said business end.
